(19) United States
(12) Patent Application Publication
(10) Pub. No.: US 2025/0197380 A1
(43) Pub. Date: Jun. 19, 2025

(54) PHARMACEUTICAL INTERMEDIATE AND SYNTHESIS METHOD, AND ISOQUINOLINE DERIVATIVE AND SYNTHESIS METHOD THEREFOR

(71) Applicant: BANGENTAI(SHANDONG) BIOSCIENCES GROUP SHARE CO., LTD., Jinan (CN)

(72) Inventors: Yumeng WEI, Jinan (CN); Limeng SUN, Jinan (CN); Chao DENG, Jinan (CN)

(21) Appl. No.: 18/849,568

(22) PCT Filed: Nov. 24, 2022
(86) PCT No.: PCT/CN2022/134144

§ 371 Date: Sep. 23, 2024

(30) Foreign Application Priority Data
Mar. 21, 2022 (CN) 202210278645.6

(51) Int. Cl. C07D 405/12; B01J 23/44; C07D 217/22

(52) U.S. Cl. CPC C07D 405/12; C07D 217/22; B01J 23/44

(57) ABSTRACT

The present invention belongs to the technical field of drug synthesis, relating to the synthesis of drugs for the prevention or treatment of diseases such as Alzheimer's disease and Parkinson's disease. Specifically, it relates to pharmaceutical intermediates and synthesis methods and isoquinoline derivatives and synthesis methods therefor. The pharmaceutical intermediate has a chemical structure shown in Formula II, wherein X is a halogen, and R is an amino protecting group. The synthesis of the pharmaceutical intermediate is simple, operates under mild conditions, and results in a high yield. Additionally, the pharmaceutical intermediate can be used to prepare isoquinoline derivatives.

The present invention claims priority to the Chinese patent application No. 202210278645.6, filed on Mar. 21, 2022, with the title “Pharmaceutical Intermediate and Synthesis Method, Isoquinoline Derivative and Synthesis Method Therefor.” The entire content of the Chinese application is incorporated herein by reference.

### TECHNICAL FIELD

The present invention belongs to the technical field of drug synthesis, relating to the synthesis of drugs for the prevention or treatment of diseases such as Alzheimer's disease and Parkinson's disease. Specifically, it relates to pharmaceutical intermediates and synthesis methods and isoquinoline derivatives and synthesis methods therefor.

### BACKGROUND

The disclosure of the background art section is intended solely to enhance the understanding of the general background of the present invention and should not necessarily be regarded as an admission or implied acknowledgment that the information constitutes prior art known to those skilled in the art.

Studies have shown that alfuzosin, terazosin, and isoquinoline derivatives not only have effects on the treatment or prevention of Parkinson's disease but also have effects on the prevention or treatment of Alzheimer's disease.

However, after research into the currently available technical literature, the inventors found no documented chemical synthesis methods for the described isoquinoline derivatives.

### SUMMARY

Isoquinoline derivatives described in the present invention are structurally very similar to alfuzosin, with the difference being that the main ring of the isoquinoline derivatives is isoquinoline, while the main ring of alfuzosin is quinazoline.

Through the inventor's research, it has been found that the synthesis routes for alfuzosin are primarily divided into three types, as follows:

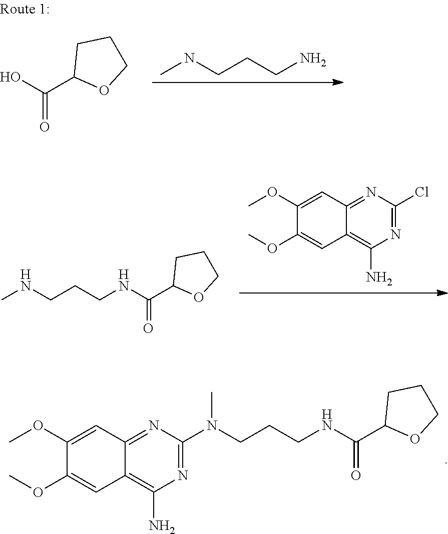

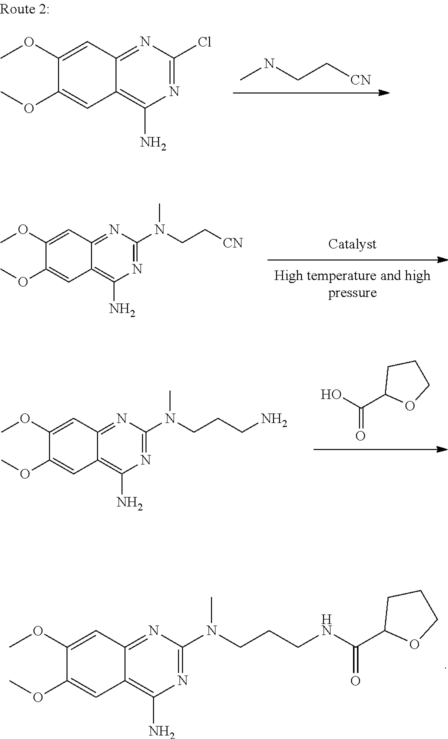

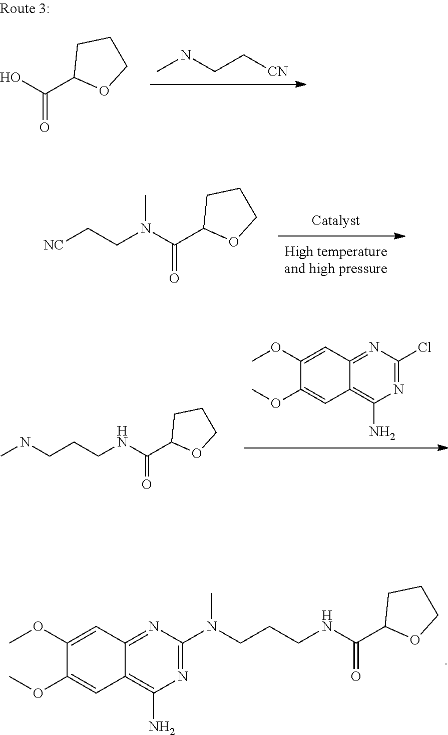

Among them, Route 1 is the first publicly disclosed process route since the disclosure of alfuzosin and is currently the more conventional process route for large-scale production of alfuzosin.

However, when using Route 1 of alfuzosin to synthesize the isoquinoline derivatives described in the present invention, the problem arises that the following structural formula is required as a starting material:

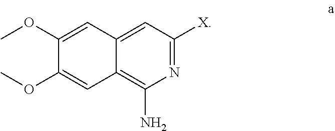

However, the compounds of the above chemical structure are not existing compounds.

Thus, the present invention can only be synthesized using the following chemical structure:

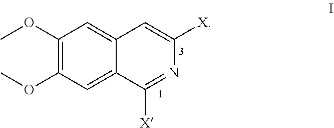

When using the above raw material to synthesize isoquinoline derivatives, it is necessary to substitute the halogen atoms (such as fluorine, chlorine, bromine, iodine, etc.) at the 1 and 3 positions of the isoquinoline ring with amines. However, compared to the quinazoline ring structure, the 5-position of the isoquinoline ring is a carbon atom, which results in a completely different electron cloud distribution from that of quinazoline. Moreover, the electron cloud distribution of the isoquinoline ring is unfavorable for amine substitution at the 1 and 3 positions, especially when methoxy groups are connected at the 6 and 7 positions, which further hinders amine substitution at the 1 and 3 positions.

Additionally, the inventors conducted the following experiments: first, the compound shown in Formula I was converted into compound a, and when compound a was reacted with N-methyl-N′-tetrahydrofuran-2-carbonylpropane diamine (i.e. tetrahydrofuran-2-carboxylicacid(3-methylamino-propyl)-amide), the desired product could not be obtained. The main reason is that the reaction temperature of compound a with N-methyl-N′-tetrahydrofuran-2-carbonylpropane diamine is too high, which leads to the consumption of the amino group at the 1 position, so the isoquinoline derivatives described in the invention cannot be obtained.

In summary, based on the existing technology, the synthesis method for the isoquinoline derivatives described in the present invention cannot be obtained.

The objective of the present invention is to provide a pharmaceutical intermediate and a synthesis method, as well as a synthesis method for isoquinoline derivatives. The synthesis of the pharmaceutical intermediate in the present invention is simple to operate, has mild conditions, and yields a high output. Furthermore, the pharmaceutical intermediate can be used to prepare isoquinoline derivatives.

To achieve the above objective, the technical solutions of the present invention are as follows:
in a first aspect, there is provided a pharmaceutical intermediate with a chemical structure shown in Formula II:

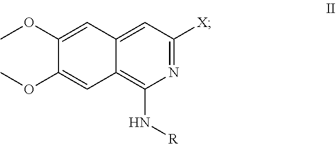

wherein, X is a halogen, such as fluorine, chlorine, bromine, iodine, etc.; R is an amino protecting group, such as benzyl, substituted benzyl, diphenylmethyl, substituted diphenylmethyl, triphenylmethyl, substituted triphenylmethyl, tert-butoxycarbonyl, etc.

In a second aspect, there is provided a method for synthesizing a pharmaceutical intermediate, including reacting a compound shown in Formula I with an amino protecting agent (R—NH₂) to obtain a compound shown in Formula II, according to the following reaction scheme:

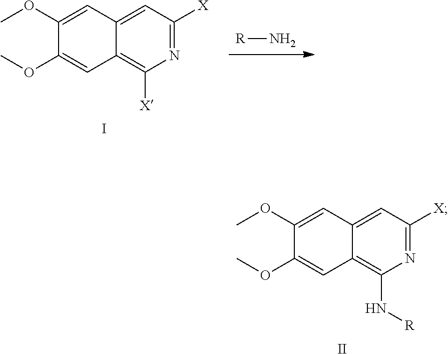

wherein X and R are selected as described above, and X′ is a halogen, such as fluorine, chlorine, bromine, iodine, etc.

The inventors have discovered that due to the electron cloud distribution of isoquinoline and the presence of methoxy groups at positions 6 and 7, which are unfavorable for amine substitution at positions 1 and 3, when the compound shown in Formula I reacts directly with ammonia, the reaction requires a temperature of 180° C. and microwave heating. To solve this issue, the present invention utilizes R—NH₂ to react, which lowers the required reaction temperature, allowing the reaction to proceed with conventional heating.

Since compound a is not an existing compound, and the compound shown in Formula II can be obtained by protecting the amino group of compound a, in a third aspect, the present invention further provides a raw material compound with a chemical structure shown in Formula a:

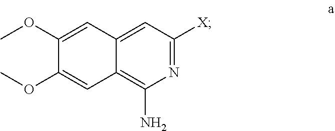

wherein X is a halogen, such as fluorine, chlorine, bromine, iodine, etc.

In a fourth aspect, there is provided a method for synthesizing isoquinoline derivatives, comprising reacting a compound shown in Formula I as a raw material to obtain a compound shown in Formula IV (isoquinoline derivative) according to the following reaction scheme:

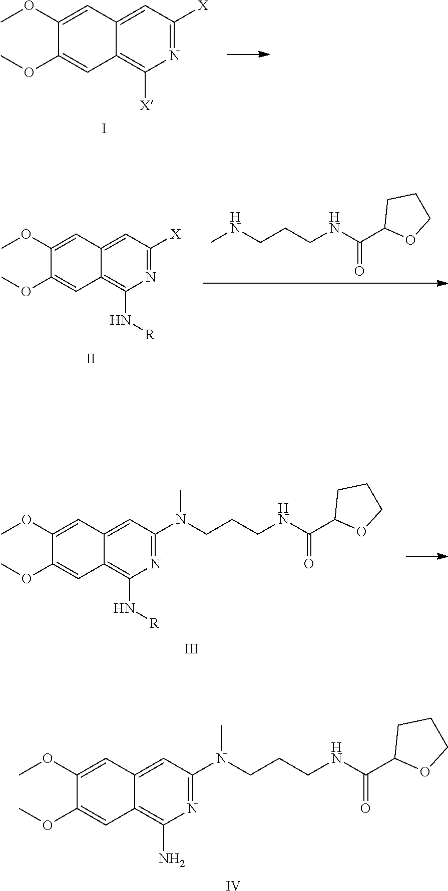

wherein X, X′, and R are selected as described above. In preparing a compound shown in Formula III from a compound shown in Formula II, ligands, catalysts, and bases are added for catalysis.

Firstly, the technical solution of the present invention allows for the preparation of the pharmaceutical intermediate (the compound shown in Formula II) under milder conditions.

Secondly, because the reactivity of the amination reaction at the 3-position is lower than that at the 1-position, the required reaction conditions are more stringent than those for the 1-position amination. When the 1-position of isoquinoline is a primary amino group, performing an amination reaction at the 3-position can lead to side reactions involving the primary amino group at the 1-position, making it impossible to prepare isoquinoline derivatives. Therefore, the present invention utilizes a pharmaceutical intermediate in which the amino group at the 1-position is protected by an amino protecting group. This approach prevents side reactions of the amino group at the 1-position, ultimately allowing the successful preparation of isoquinoline derivatives. Meanwhile, the research in the present invention indicates that achieving the amination reaction at the 3-position requires temperatures of 220° C. or higher. Moreover, the selectivity is low, and numerous by-products are formed, resulting in an extremely low yield (below 2%) of the target compound (i.e., the compound shown in Formula III). By adding ligands, catalysts, and bases for catalysis, the present invention not only lowers the reaction temperature but also significantly increases the yield of the compound shown in Formula III.

Thirdly, the technical solution provided by the present invention involves fewer reactions and simpler operational steps.

From the description of the above technical solutions, it is evident that a person skilled in the art cannot obtain the isoquinoline derivatives based on the existing technology. Therefore, in a fifth aspect, the present invention provides an isoquinoline derivative, which is the compound shown in Formula IV obtained through the aforementioned synthesis method.

### Beneficial Effects of the Present Invention

1. The present invention provides a pharmaceutical intermediate that can be used to prepare isoquinoline derivatives, which have effects on improving mitochondrial metabolism, degrading various pathological protein accumulations, and improving vascular endothelial function, and can be used for Alzheimer's disease and related complications.

2. The method for preparing the pharmaceutical intermediate in the present invention uses conventional heating and does not require harsh reaction conditions.

3. The isoquinoline derivatives prepared using the pharmaceutical intermediate provided by the present invention have high purity and yield.

### BRIEF DESCRIPTION OF THE DRAWINGS

The accompanying drawings to the specification, which form part of the present invention, are used to provide a further understanding of the present invention, and the illustrative examples of the present invention and the description thereof are used to explain the present invention and are not unduly limiting the present invention.

FIG. 1 is a graph showing the test results of the wall-climbing behavior in rats of a Parkinson's disease model induced by injecting 6-hydroxydopamine (6-OHDA) into specific brain regions, treated with Compound IV, as described in Example 16 of the present invention.

FIG. 2 is a graph showing the test results of motor activity in rats of a Parkinson's disease model induced by injecting 6-OHDA into specific brain regions, treated with Compound IV, as described in Example 16 of the present invention.

FIG. 3 is a graph showing the test results of grip strength and neurological balance ability in mice of a Parkinson's disease model induced by intraperitoneal injection of 1-methyl-4-phenyl-1,2,3,6-tetrahydropyridine (MPTP), treated with Compound IV, as described in Example 17 of the present invention.

FIG. 4 is a graph showing the results of the number of surviving dopaminergic neurons in mice of a Parkinson's disease model induced by intraperitoneal injection of MPTP, treated with Compound IV, as described in Example 17 of the present invention.

### DESCRIPTION OF THE PREFERRED EMBODIMENTS

It should be noted that the following detailed descriptions are all illustrative and intended to provide further clarification of the present invention. Unless otherwise specified, all technical and scientific terms used herein have the same meaning as commonly understood by one of ordinary skill in the art to which this invention belongs.

It should be noted that the terminology used here is for the purpose of describing particular embodiments only and is not intended to be limiting of the exemplary embodiments of the invention. As used herein, unless explicitly stated otherwise, the singular form is intended to include the plural form as well. Additionally, it should be understood that when the terms “comprising” and/or “including” are used in this specification, they indicate the presence of features, steps, operations, devices, components, and/or their combinations.

To achieve the synthesis of isoquinoline derivatives, the present invention proposes a pharmaceutical intermediate, synthesis method therefor, and a method for synthesizing isoquinoline derivatives.

In a typical embodiment of the present invention, a pharmaceutical intermediate is provided with the chemical structure shown in Formula II:

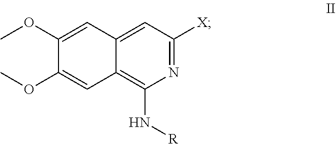

wherein, X is a halogen, such as fluorine, chlorine, bromine, iodine, etc.; R is an amino protecting group, such as benzyl, substituted benzyl, diphenylmethyl, substituted diphenylmethyl, triphenylmethyl, substituted triphenylmethyl, tert-butoxycarbonyl, etc. The substituted benzyl is benzyl substituted by alkoxy, halogen, alkyl, or the like. The substituted diphenylmethyl is diphenylmethyl substituted by alkoxy, halogen, alkyl, or the like. The substituted triphenylmethyl is triphenylmethyl substituted by alkoxy, halogen, alkyl, or the like.

In some examples of this embodiment, the amino protecting group is a benzyl group substituted by alkoxy or alkyl, such as p-methoxybenzyl, 2-methylbenzyl, 3,4-dimethoxybenzyl, or 2,4-dimethoxybenzyl, ect.

In a second embodiment of the present invention, a method for synthesizing the pharmaceutical intermediate described above is provided, which includes reacting the compound shown in Formula I with an amino protecting agent according to the following reaction scheme to obtain the compound shown in Formula II:

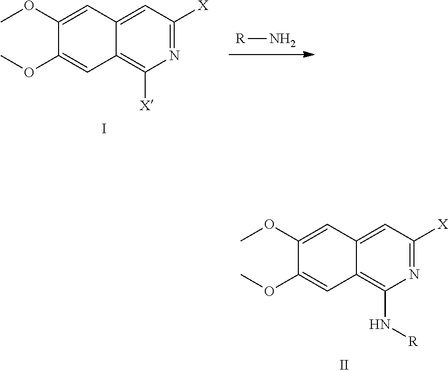

wherein X and R are selected as described above, and X′ is a halogen, such as fluorine, chlorine, bromine, iodine, etc.

The present invention utilizes R—NH₂ to react, which lowers the required reaction temperature, allowing the reaction to proceed with conventional heating.

Research has shown that the type of amino protecting group affects the yield of the pharmaceutical intermediate. When the amino protecting group is an alkoxy-substituted benzyl or alkyl-substituted benzyl (such as p-methoxybenzyl, 2-methylbenzyl, 3,4-dimethoxybenzyl, or 2,4-dimethoxybenzyl), the yield of the pharmaceutical intermediate is higher.

In some examples of this embodiment, a reaction solvent is selected from one or more of a group consisting of N-methylpyrrolidone (NMP), N,N-dimethylformamide (DMF), and dimethyl sulfoxide (DMSO).

In some examples of this embodiment, a reaction temperature is 100-160° C., preferably 100-130° C.

In some examples of this embodiment, a mass/volume ratio of the compound shown in Formula I to the reaction solvent is 1:(5-30) g/mL, preferably 1:10 g/mL.

In some examples of this embodiment, a molar ratio of the compound shown in Formula I to the amino protecting agent is 1:(2-5), preferably 1:(2.5-4).

A purification method for the pharmaceutical intermediate (the compound shown in Formula II) comprises adding water to a post-reaction material to quench the reaction, extracting the quenched material, washing, drying, and evaporating the solvent from the extracted organic phase to obtain the purified pharmaceutical intermediate.

In a third embodiment of the present invention, a raw material compound is provided, having a chemical structure shown in Formula a:

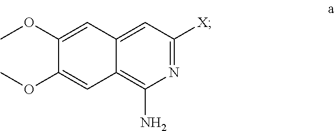

wherein X is a halogen, such as fluorine, chlorine, bromine, iodine, etc.

In a fourth embodiment of the present invention, a method for synthesizing isoquinoline derivatives is provided, which includes using the compound shown in Formula I as a raw material to obtain the compound shown in Formula IV (isoquinoline derivative) according to the following reaction scheme:

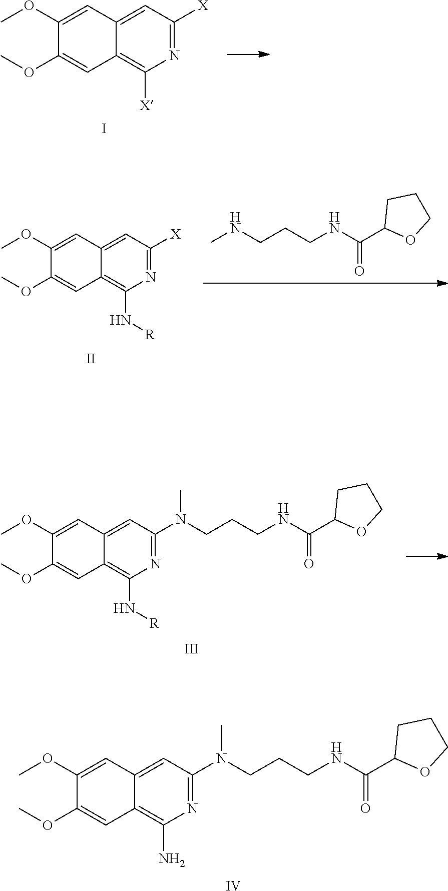

wherein, in preparing a compound shown in Formula III from a compound shown in Formula II, a ligand, a catalyst, and a base are added for catalysis.

The present invention not only provides milder reaction conditions for synthesizing the compounds shown in Formula II and Formula III but also achieves a higher yield for the compound shown in Formula III, with fewer operational steps.

The route for converting the compound shown in Formula I to the compound shown in Formula II can be either Route One or Route Two.

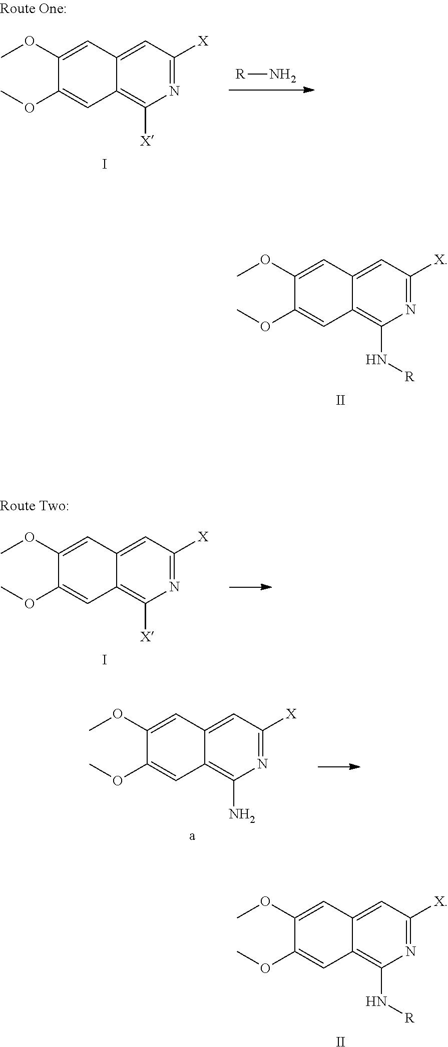

When Route One is used, the process of converting the compound shown in Formula I to the compound shown in Formula II can refer to the second embodiment mentioned above.

In some examples of this embodiment, during the process of converting the compound shown in Formula II to the compound shown in Formula III, the catalyst used is a palladium catalyst or a copper catalyst. The type of catalyst affects the yield of the compound shown in Formula III. Research shows that using the palladium catalyst results in a higher yield. The palladium catalyst can be one or a combination of the following: PdCl₂, Pd(PPh₃)₄ (i.e., tetrakis(triphenylphosphine)palladium), Pd(OAc)₂ (i.e., Palladium(II) acetate), Pd₂(dba)₃ (i.e., tris(dibenzylideneacetone)dipalladium), Pd(dba)₂ (i.e., bis(dibenzylideneacetone)palladium), PdCl₂(cod) (i.e., (1,5-Cyclooctadiene)palladium chloride), [Pd(allyl)Cl]₂ (i.e., allylpalladium(II) chloride dimer), PdCl₂ (CH₃CN)₂ (i.e., bis(acetonitrile)palladium(II) chloride), Pd(acac)₂ (i.e., palladium(II) acetylacetonate), Pd(PPh₃)₂Cl₂ (i.e., bis(triphenylphosphine)palladium(II) chloride), Pd(Dppf)₂Cl₂ (i.e., 1,1′-bis(diphenylphosphino)ferrocene palladium dichloride), PdCl₂[P(o-Tol)₃](i.e., trans-dichlorobis(tri-o-tolylphosphine)palladium).

In some examples of this embodiment, during the process of converting the compound shown in Formula II to the compound shown in Formula III, the ligand used is one or more selected from: PPh₃ (i.e., triphenylphosphine), P(o-tolyl)₃ (i.e., tris(o-tolyl)phosphine), P(t-Bu)₃ (i.e., tris(tert-butyl)phosphine), P(t-Bu)₃·HBF₄ (i.e., tris(tert-butyl)phosphine tetrafluoroborate), PCy₃ (i.e., tricyclohexylphosphine), n-BuP(Ad)₂, BINAP (i.e., 2,2′-bis(diphenylphosphino)-1,1′-binaphthyl), Xantphos (i.e., 4,5-bis(diphenylphosphino)-9,9-dimethylxanthene), DPEPhos (i.e., bis(2-diphenylphosphinophenyl) ether), Dppf (i.e., 1,1′-bis(diphenylphosphino)ferrocene), CyPFt-Bu, Dppp (i.e., 1,3-bis(diphenylphosphino)propane), JohnPhos (i.e., 2-(di-tert-butylphosphino)biphenyl), CyJohnPhos (i.e., 2-(dicyclohexylphosphino)biphenyl), DavePhos (i.e., 2-(dicyclohexylphosphino)-2′-(N,N-dimethylamino)biphenyl), RuPhos (i.e., 2-(dicyclohexylphosphino)-2′,6′-diisopropoxybiphenyl), SPhos (i.e., 2-(dicyclohexylphosphino)-2′,6′-dimethoxybiphenyl), Xphos (i.e., 2-(dicyclohexylphosphino)-2′,4′,6′-triisopropylbiphenyl), BrettPhos (i.e., 2-(dicyclohexylphosphino)-3,6-dimethoxy-2′,4′,6′-triisopropylbiphenyl), t-BuXphos (i.e., 2-(di-tert-butylphosphino)-2′,4′,6′-triisopropylbiphenyl), t-BuBrettPhos (i.e., 2-(di-t-butylphosphino)-3,6-dimethoxy-2′4′6′-tri-i-propyl-1,1′-biphenyl), Me₄t-BuXphos (i.e., 2-(di-tert-butylphosphino)-3,4,5,6-tetramethyl-2′,4′,6′-triisopropylbiphenyl), Bippyphos (i.e., 5-(di-tert-butylphosphino)-1′,3′,5′-triphenyl-1′H-[1,4′]bipyrazole), MorDalPhos, and IPr HCl (i.e., 1,3-bis(2,6-diisopropylphenyl)imidazolium chloride).

In some examples of this embodiment, during the process of converting the compound shown in Formula II to the compound shown in Formula III, the base used is sodium tert-butoxide, cesium carbonate, potassium tert-butoxide, potassium carbonate, potassium phosphate, lithium bis(trimethylsilyl)amide, DBU (1,8-Diazabicyclo[5.4.0]undec-7-ene), or MTBD (7-Methyl-1,5,7-triazabicyclo[4.4.0]dec-5-ene).

Research indicates that the combined use of the above catalysts, ligands, and bases yields better results. For example, using a combination of Pd₂(dba)₃, DavePhos and Cs₂CO₃, the yield of the compound shown in Formula III can reach 10%; using a combination of Pd(OAc)₂, Dppf and Cs₂CO₃, the yield can reach 22%; using a combination of Pd(OAc)₂, BINAP and Cs₂CO₃, the yield can reach 36%; using a combination of Pd(OAc)₂, BINAP and t-BuONa, the yield can reach 37%; using a combination of Pd₂(dba)₃, RuPhos and t-BuONa, the yield can reach 53%. However, using BrettPhos-Pd-G₃/Cs₂CO₃ results in a lower yield.

In some examples of this embodiment, during the process of converting the compound shown in Formula II to the compound shown in Formula III, the reaction temperature is 90-130° C., preferably 95-110° C.

In some example of this embodiment, during the process of converting the compound shown in Formula II to the compound shown in Formula III, the solvent used is an organic solvent with a boiling point higher than 90° C. Research shows that when using low-boiling-point organic solvents (below 90° C.), such as tetrahydrofuran, the reaction must be sealed, and meanwhile microwave heating is required. The present invention found that after combining the above catalysts, ligands, and bases, using high-boiling-point organic solvents (above 90° C.), such as toluene, 1,4-dioxane, N,N-dimethylformamide (DMF), and dimethyl sulfoxide (DMSO), allows the reaction to proceed through simple heating.

In some examples of this embodiment, during the process of converting the compound shown in Formula II to the compound shown in Formula III, a mass/volume ratio of the compound shown in Formula II to the reaction solvent is 1:(5-100) g/mL, preferably 1:(10-20) g/mL.

In some examples of this embodiment, during the process of converting the compound shown in Formula II to the compound shown in Formula III, a molar ratio of the compound shown in Formula II to N-methyl-N′-tetrahydrofuranformylpropylenediamine (i.e., tetrahydrofuran-2-carboxylicacid(3-methylamino-propyl)-amide) is 1:(1-5), preferably 1:(1.5-3).

In some examples of this embodiment, during the process of converting the compound shown in Formula II to the compound shown in Formula III, a molar ratio of the compound shown in Formula II to the catalyst is 1:(0.01-0.5), preferably 1:(0.05-0.2).

In some examples of this embodiment, during the process of converting the compound shown in Formula II to the compound shown in Formula III, a molar ratio of the compound shown in Formula II to the ligand is 1:(0.02-1.0), preferably 1:(0.05-0.4).

In some examples of this embodiment, during the process of converting the compound shown in Formula II to the compound shown in Formula III, a molar ratio of the compound shown in Formula II to the base is 1:(1-4), preferably 1:(1.2-2).

In some examples of this embodiment, during the process of converting the compound shown in Formula III to the compound shown in Formula IV, a reaction temperature is 0-70° C., preferably 0-40° C.

In some examples of this embodiment, during the process of converting the compound shown in Formula III to the compound shown in Formula IV, the reaction solvent is trifluoroacetic acid, triethylsilane, methanesulfonic acid, trifluoromethanesulfonic acid, a mixture of trifluoroacetic acid and dichloromethane, a mixture of methanesulfonic acid and dichloromethane, or a mixture of triethylsilane and trifluoroacetic acid, etc.

In some examples of this embodiment, during the process of converting the compound shown in Formula III to the compound shown in Formula IV, a mass/volume ratio of the compound shown in Formula III to the reaction solvent is 1(5-100) g/mL, preferably 1(5-20) g/mL.

The preferred steps of the present invention are as follows:

(1) the starting material, 1,3-dichloro-6,7-dimethoxyisoquinoline (the compound shown in Formula I), is added to a reaction solvent and stirred to dissolve. Then, an amino protecting agent is added, and the temperature is raised to 90-160° C. After the reaction is complete, the compound shown in Formula II is obtained.

(2) The compound shown in Formula II is subjected to a Buchwald-Hartwig coupling reaction with N-methyl-N′-tetrahydrofuranformylpropylenediamine (i.e., tetrahydrofuran-2-carboxylicacid(3-methylamino-propyl)-amide) or its salt to obtain the compound shown in Formula III.

(3) The compound shown in Formula III is added to a reaction solvent, and the temperature is controlled at 0-70° C. to remove the amino protecting group, yielding the desired product.

In the fourth embodiment of the present invention, an isoquinoline derivative is provided, which is the compound shown in Formula IV obtained by the above method for synthesizing isoquinoline derivatives.

To enable those skilled in the art to better understand the technical solutions of the present invention, the following will provide a detailed description of the technical solutions in conjunction with specific examples.

### Example 1: Synthesis of the Compound Shown in Formula II (in this Example, R=p-Methoxybenzyl)

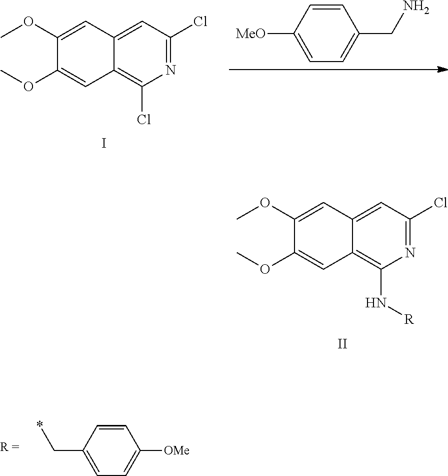

To a reaction flask, compound shown in Formula I (10 g) and N-methylpyrrolidone (100 mL) were added and stirred to dissolve. p-Methoxybenzylamine (15.9 g) was added, and the temperature was raised to 120° C. The reaction was run for 4 hours, monitored by TLC (petroleum ether/ethyl acetate=2/1). Upon completion, water (300 mL) was added to quench the reaction. The mixture was extracted three times with dichloromethane (200 mL×3), and the organic layers were combined. The organic phase was washed once with saturated brine (300 mL), dried over anhydrous sodium sulfate, and the solvent was evaporated. The product was purified by column chromatography (SiO₂) to obtain 12.8 g of the compound shown in Formula II with a yield of 92.1%.

¹H NMR results for the compound shown in Formula II:

¹H NMR: (400 MHz, DMSO-d₆) δ8.08-7.88 (m, 1H), 7.64 (s, 1H), 7.32 (d, J=8.8 Hz, 2H), 7.14 (s, 1H), 6.93-6.80 (m, 3H), 4.62 (d, J=6.0 Hz, 2H), 3.87 (d, J=3.6 Hz, 6H), 3.79-3.61 (m, 3H).

LC-MS (C₁₉H₁₉ClN₂O₃): 359.1 [M+H]⁺.

### Example 2: Synthesis of the Compound Shown in Formula II (in this Example, R=2-Methylbenzyl)

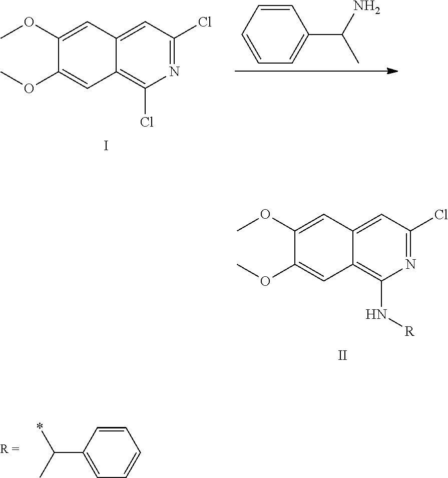

To a reaction flask, compound shown in Formula I (1 g) and N,N-dimethylformamide (DMF) were added and stirred to dissolve. 2-Methylbenzylamine (1.88 g) was added, and the temperature was raised to 120° C. The reaction was run for 4 hours, monitored by TLC (petroleum ether/ethyl acetate=3/1). Upon completion, water (30 mL) was added to quench the reaction. The mixture was extracted three times with dichloromethane (20 mL×3), and the organic layers were combined. The organic phase was washed once with saturated brine (30 mL), dried over anhydrous sodium sulfate, and the solvent was evaporated. The product was purified by column chromatography (SiO₂) to obtain 1.01 g of the compound shown in Formula II with a yield of 76.0%.

LC-MS (C₁₉H₁₉ClN₂O₂): 343.0 [M+H]⁺.

### Example 3: Synthesis of the Compound Shown in Formula II (in this Example, R=3,4-Dimethoxybenzyl)

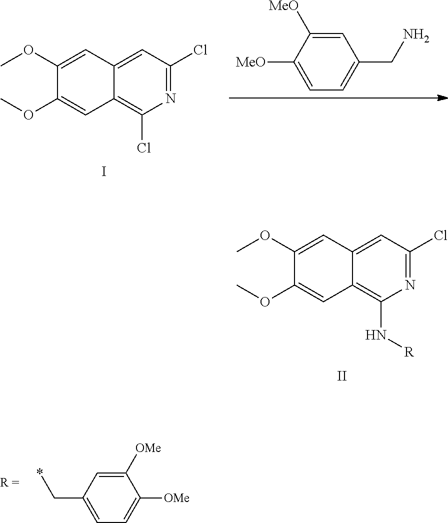

To a reaction flask, compound shown in Formula I (1 g) and N-methylpyrrolidone (10 mL) were added and stirred to dissolve. 3,4-Dimethoxybenzylamine (2.58 g) was added, and the temperature was raised to 120° C. The reaction was run for 4 hours, monitored by TLC (petroleum ether/ethyl acetate=2/1). Upon completion, water (50 mL) was added to quench the reaction. The mixture was extracted three times with dichloromethane (30 mL×3), and the organic layers were combined. The organic phase was washed once with saturated brine (30 mL), dried over anhydrous sodium sulfate, and the solvent was evaporated. The product was purified by column chromatography (SiO₂) to obtain 1.29 g of the compound shown in Formula II with a yield of 85.5%.

LC-MS (C₂₀H₂₁ClN₂O₄): 389.1 [M+H]⁺.

### Example 4: Synthesis of the Compound Shown in Formula II (in this Example, R=2,4-Dimethoxybenzyl)

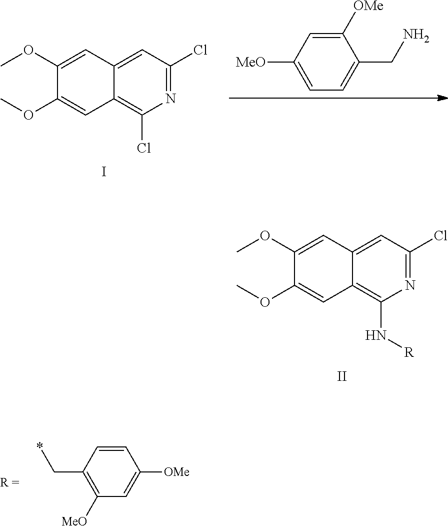

To a reaction flask, compound shown in Formula I (1 g) and N-methylpyrrolidone (10 mL) were added and stirred to dissolve. 2,4-Dimethoxybenzylamine (2.58 g) was added, and the temperature was raised to 120° C. The reaction was run for 4 hours, monitored by TLC (petroleum ether/ethyl acetate=2/1). Upon completion, water (50 mL) was added to quench the reaction. The mixture was extracted three times with dichloromethane (30 mL×3), and the organic layers were combined. The organic phase was washed once with saturated brine (30 mL), dried over anhydrous sodium sulfate, and the solvent was evaporated. The product was purified by column chromatography (SiO₂) to obtain 1.35 g of the compound shown in Formula II with a yield of 89.6%.

LC-MS (C₂₀H₂₁ClN₂O₄): 389.1 [M+H]⁺.

### Example 5: Synthesis of the Compound Shown in Formula II (in this Example, R=p-Methoxybenzyl)

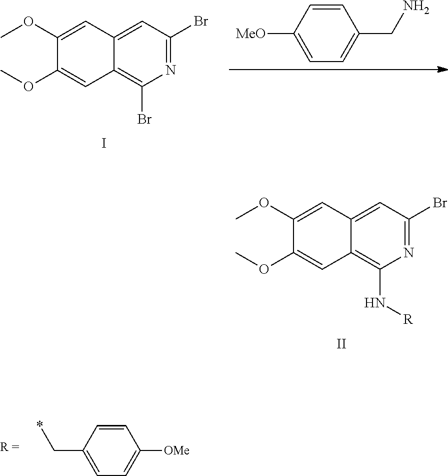

To a reaction flask, compound shown in Formula I (1 g) and N-methylpyrrolidone (10 mL) were added and stirred to dissolve. p-Methoxybenzylamine (1.58 g) was added, and the temperature was raised to 120° C. The reaction was run for 4 hours, monitored by TLC (petroleum ether/ethyl acetate=2/1). Upon completion, water (300 mL) was added to quench the reaction. The mixture was extracted three times with dichloromethane (200 mL×3), and the organic layers were combined. The organic phase was washed once with saturated brine (300 mL), dried over anhydrous sodium sulfate, and the solvent was evaporated. The product was purified by column chromatography (SiO₂) to obtain 0.98 g of the compound shown in Formula II with a yield of 84.3%.

In this example, p-methoxybenzylamine can be replaced by 2-methylbenzylamine, 2,4-dimethoxybenzylamine, or 3,4-dimethoxybenzylamine.

### Example 6: Synthesis of the Compound Shown in Formula III (in this Example, R=p-Methoxyphenyl)

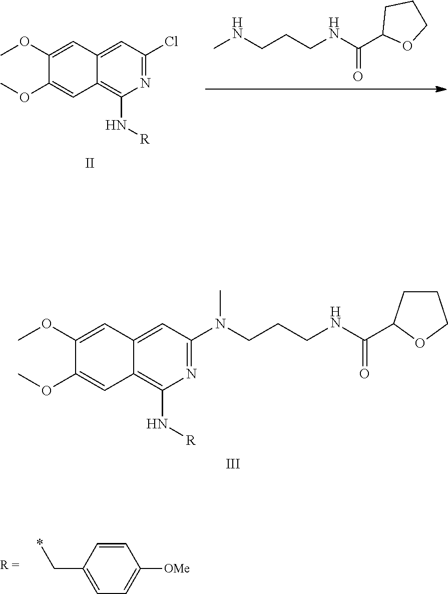

In a reaction flask, toluene (30 mL), the compound shown in Formula II (2.00 g), N-methyl-N′-tetrahydrofuranformylpropylenediamine (2.08 g), sodium tert-butoxide (1.07 g), Pd₂(dba)₃ (1.02 g), and RuPhos (1.04 g) were added. The reaction mixture was heated to 100° C. under nitrogen protection for 3 hours. After completion, the reaction was filtered, and the filtrate was concentrated under vacuum. The product was purified by column chromatography (SiO₂), eluted with a mixture of petroleum ether and ethyl acetate. The eluent was concentrated to obtain 3.00 g of the compound shown in Formula III with a yield of 52.9%.

¹H NMR results for R=p-methoxybenzyl:

¹H NMR: (400 MHz, CDCl₃) δ 7.92-7.78 (m, 1H), 7.41-7.34 (m, 1H), 7.03 (d, J=8.8 Hz, 2H), 6.94-6.77 (m, 3H), 4.46-4.25 (m, 1H), 4.18-4.07 (m, 2H), 4.04-4.00 (m, 1H), 4.06-4.00 (m, 1H), 3.98 (s, 2H), 3.93-3.89 (m, 4H), 3.83 (s, 2H), 3.76-3.66 (m, 2H), 3.41-3.22 (m, 3H), 3.02-2.87 (m, 3H), 2.00-1.97 (m, 1H), 1.99-1.73 (m, 10H).

LC-MS (C₂₈H₃₆N₄O₅): 509.1 [M+H]⁺, 531.1 [M+Na]⁺.

### Example 7: Synthesis of the Compound Shown in Formula III (in this Example, R=2-Methylbenzyl)

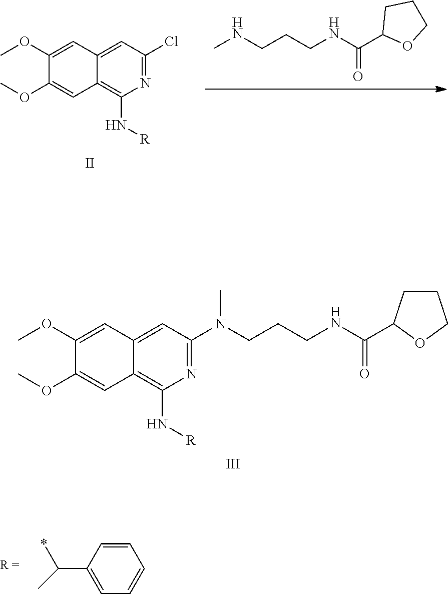

In a reaction flask, toluene (2.5 mL), the compound shown in Formula II (0.5 g), N-methyl-N′-tetrahydrofuranformylpropylenediamine (0.68 g), sodium tert-butoxide (0.54 g), Pd₂(dba)₃ (0.08 g), and RuPhos (0.06 g) were added. The reaction mixture was heated to 100° C. under nitrogen protection for 3 hours. After completion, the reaction was filtered, and the filtrate was concentrated under vacuum. The product was purified by column chromatography (SiO₂), eluted with a mixture of petroleum ether and ethyl acetate. The eluent was concentrated to obtain 0.26 g of the compound shown in Formula III with a yield of 36.2%.

LC-MS (C₂₈H₃₆N₄O₄): 493.3 [M+H]⁺.

### Example 8: Synthesis of the Compound Shown in Formula III (in this Example, R=2,4-Dimethoxybenzyl)

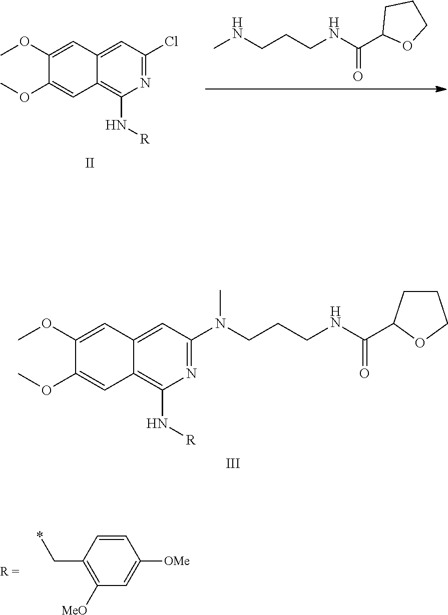

In a reaction flask, 1,4-dioxane (10 mL), the compound shown in Formula II (1.1 g), N-methyl-N′-tetrahydrofuranformylpropylenediamine (0.72 g), cesium carbonate (1.85 g), Pd(OAc)₂ (0.07 g), and RuPhos (0.26 g) were added. The reaction mixture was heated to 100° C. under nitrogen protection for 3 hours. After completion, the reaction was filtered, and the filtrate was concentrated under vacuum. The product was purified by column chromatography (SiO₂), eluted with a mixture of petroleum ether and ethyl acetate. The eluent was concentrated to obtain 1.02 g of the compound shown in Formula III with a yield of 67.0%.

LC-MS (C₂₉H₃₈N₄O₆): 539.3 [M+H]⁺.

### Example 9: Synthesis of the Compound Shown in Formula III (in this Example, R=3,4-Dimethoxybenzyl)

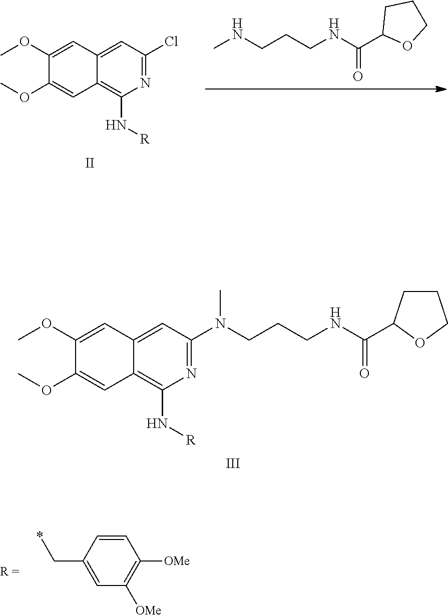

In a reaction flask, 1,4-dioxane (20 mL), the compound shown in Formula II (1.0 g), N-methyl-N′-tetrahydrofuranformylpropylenediamine (0.68 g), cesium carbonate (1.77 g), Pd(OAc)₂ (0.06 g), and SPhos (0.21 g) were added. The reaction mixture was heated to 100° C. under nitrogen protection for 3 hours. After completion, the reaction was filtered, and the filtrate was concentrated under vacuum. The product was purified by column chromatography (SiO₂), eluted with a mixture of petroleum ether and ethyl acetate. The eluent was concentrated to obtain 0.91 g of the compound shown in Formula III with a yield of 65.7%.

LC-MS (C₂₉H₃₈N₄O₆): 539.3 [M+H]⁺.

### Example 10: Synthesis of the Compound Shown in Formula III (in this Example, R=p-Methoxyphenyl)

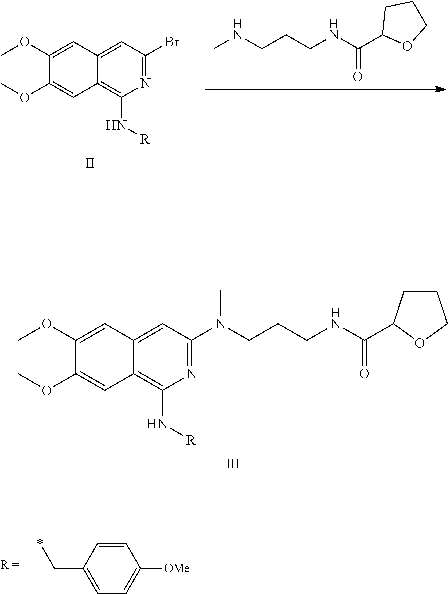

In a reaction flask, toluene (5 mL), the compound shown in Formula II (0.56 g), N-methyl-N′-tetrahydrofuranformylpropylenediamine (0.52 g), sodium tert-butoxide (0.27 g), Pd₂(dba)₃ (0.26 g), and RuPhos (0.26 g) were added. The reaction mixture was heated to 100° C. under nitrogen protection for 3 hours. After completion, the reaction was filtered, and the filtrate was concentrated under vacuum. The product was purified by column chromatography (SiO₂), eluted with a mixture of petroleum ether and ethyl acetate. The eluent was concentrated to obtain 0.42 g of the compound shown in Formula III with a yield of 59.4%.

In this example, p-methoxybenzylamine can be replaced by 2-methylbenzylamine, 2,4-dimethoxybenzylamine, or 3,4-dimethoxybenzylamine.

### Example 11: Synthesis of the Compound Shown in Formula IV (in this Example, R=p-Methoxybenzyl)

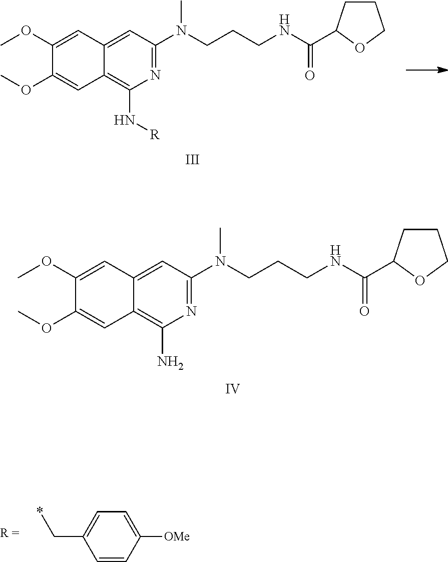

In a reaction flask, dichloromethane (20 mL) and the compound shown in Formula III (2 g) were added under nitrogen protection. Trifluoroacetic acid (4 mL) was added dropwise while keeping the temperature below 20° C. The reaction was stirred at a temperature below 20° C. for 2 hours. After completion, saturated NaHCO₃ solution was added to the reaction mixture to adjust the pH to 7-8. The mixture was extracted with dichloromethane three times, and the combined organic layers were concentrated under vacuum. The product was purified by column chromatography (SiO₂), eluted with a mixture of dichloromethane and methanol (50:1 to 5:1), to obtain 1.03 g of the compound shown in Formula IV with a yield of 67.4%.

¹H NMR: (400 MHz, CDCl₃) (8.48 (br s, 1H), 6.85 (s, 1H), 6.79 (s, 1H), 5.98 (s, 1H), 5.38 (s, 2H), 4.49 (dd, J=5.6, 8.4 Hz, 1H), 4.16-4.04 (m, 1H), 4.01-3.93 (m, 6H), 3.92-3.86 (m, 1H), 3.58-3.50 (m, 1H), 3.45-3.32 (m, 1H), 3.04 (tdd, J=4.4, 9.2, 13.6 Hz, 1H), 2.92 (s, 3H), 2.34-2.18 (m, 2H), 1.98-1.86 (m, 2H), 1.78 (tdd, J=4.8, 9.6, 14.4 Hz, 1H) LC-MS (C₂₀H₂₈N₄O₄): 389.1 [M+H]⁺.

### Example 12: Synthesis of the Compound Shown in Formula IV (in this Example, R=2-Methylbenzyl)

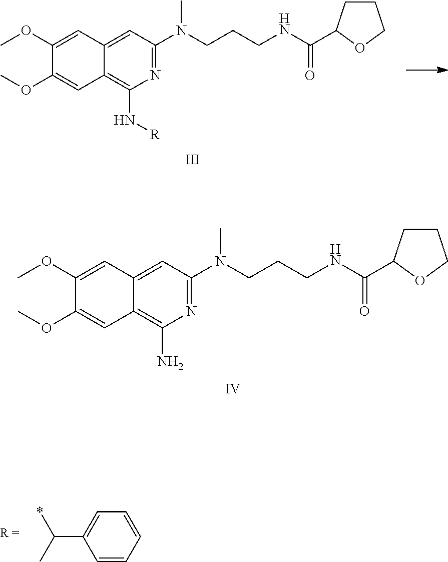

Under nitrogen protection, ethanol (2 mL) and the compound shown in Formula III (0.2 g) were added to a reaction flask. Pd/C (0.05 g) and Pd(OH)₂ (0.05 g) were added, and the system was flushed with nitrogen three times and then with oxygen three times. The reaction was heated to 50° C. and stirred for at least 16 hours. After completion, the reaction was cooled to 20-30° C. and filtered through diatomaceous earth. The solvent was evaporated under vacuum, and the residue was purified by column chromatography (SiO₂) using dichloromethane/methanol (50:1 to 5:1) as the eluent. A total of 0.09 g of the compound shown in Formula IV was obtained, with a yield of 56.2%.

### Example 13: Synthesis of the Compound Shown in Formula IV (in this Example, R=2,4-Dimethoxybenzyl)

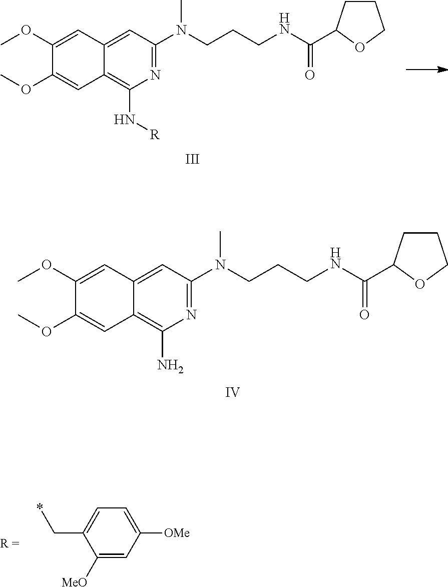

Under nitrogen protection, dichloromethane (5 mL) and the compound shown in Formula III (1.0 g) were added to a reaction flask. Trifluoroacetic acid (1.0 mL) and trifluoromethanesulfonic acid (2.0 mL) were added while keeping the temperature below 20° C. The reaction was stirred for 2 hours at below 20° C. After completion, a saturated NaHCO₃ solution was added to adjust the pH to 7-8. The mixture was extracted three times with dichloromethane, and the combined organic layers were concentrated under vacuum. The residue was purified by column chromatography (SiO₂) using dichloromethane/methanol (50:1 to 5:1) as the eluent. A total of 0.47 g of the compound shown in Formula IV was obtained, with a yield of 65.1%.

### Example 14: Synthesis of the Compound Shown in Formula IV (in this Example, R=3,4-Dimethoxybenzyl)

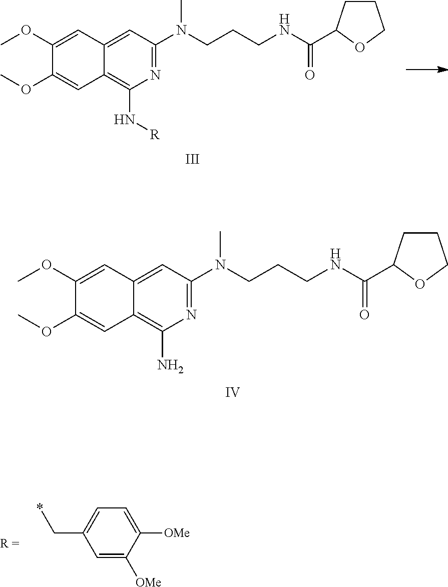

Under nitrogen protection, dichloromethane (5 mL) and the compound shown in Formula III (0.5 g) were added to a reaction flask. Trifluoroacetic acid (1.0 mL) and trifluoromethanesulfonic acid (0.5 mL) were added while keeping the temperature below 20° C. The reaction was stirred for 2 hours at below 20° C. After completion, a saturated NaHCO₃ solution was added to adjust the pH to 7-8. The mixture was extracted three times with dichloromethane, and the combined organic layers were concentrated under vacuum. The residue was purified by column chromatography (SiO₂) using dichloromethane/methanol (50:1 to 5:1) as the eluent. A total of 0.21 g of the compound shown in Formula IV was obtained, with a yield of 58.2%.

### Example 15: Salt Forms of the Compound Shown in Formula IV

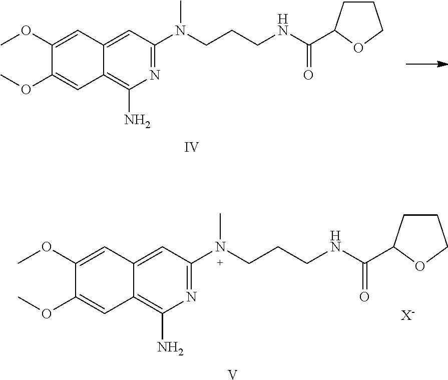

The compound shown in Formula IV can form salts as shown in Formula V, including hydrochloride, sulfate, phosphate, p-toluenesulfonate, methanesulfonate, hydrobromide, ethanesulfonate, acetate, maleate, fumarate, and succinate.

### Example 16: Effect of the Compound Shown in Formula IV on 6-OHDA Parkinson's Disease Rat Model

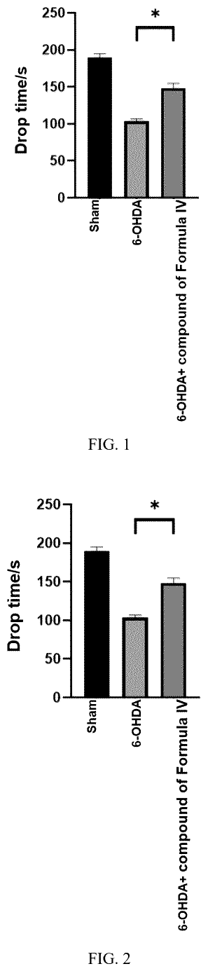

A rat Parkinson's disease model was established by injecting 6-OHDA into a specific brain region (right striatum). Two to three weeks after 6-OHDA injection, treatment with the compound shown in Formula IV was administered for two weeks. In the cylinder test, the asymmetric use of limbs during wall climbing was evaluated, as shown in FIG. 1. After 6-OHDA treatment, the use of the affected forelimb decreased, reflected by an increase in the forelimb asymmetry index, the forelimb asymmetry index=(use of unaffected limb−use of affected limb)/(use of unaffected limb+use of affected limb+use of both limbs)×100%. Treatment with the compound shown in Formula IV significantly reduced the forelimb asymmetry index.

Additionally, the rotarod test was used to evaluate motor function in rats. After 6-OHDA treatment, the time that the rats could maintain on the rotarod was significantly reduced. Treatment with the compound shown in Formula IV significantly improved this ability, as shown in FIG. 2.

### Example 17: Effect of the Compound Shown in Formula IV on MPTP Parkinson's Disease Mouse Model

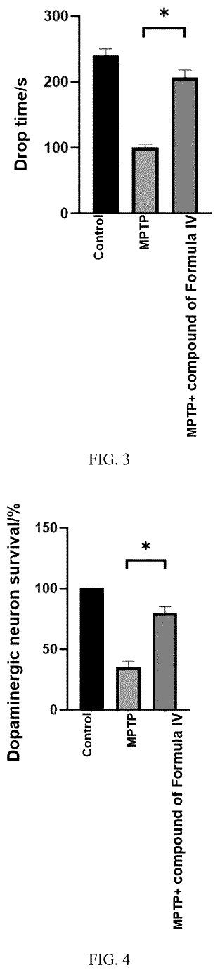

The Parkinson's disease mouse model was induced by intraperitoneal injection of MPTP, with a dose of 20 mg/kg administered intraperitoneally for seven days, followed by the Rotarod test to assess the effects of the compound. This test quantitatively measures the grip strength and neurological balance ability of the mice. After MPTP treatment, these abilities were significantly reduced, but treatment with the compound shown in Formula IV led to a significant improvement, as shown in FIG. 3. Ten mice were tested in each group.

Dopaminergic neurons in the substantia nigra pars compacta (SNpc) were stained with dopamine antibodies, and the results are shown in FIG. 4. Compared with normal mice, MPTP treatment resulted in a significant reduction in the number of dopaminergic neurons. However, in the MPTP-treated mice that were orally administered the compound shown in Formula IV, the number of dopaminergic neurons significantly increased. This indicates a significant difference in neuron survival rate (p<0.01) between the MPTP-treated mice given the compound and those not given the compound.

The above descriptions are merely preferred embodiments of the present invention and are not intended to limit the present invention. For those skilled in the art, various changes and modifications can be made to the present invention. Any modification, equivalent replacement, or improvement made within the spirit and principles of the present invention should be included within the scope of the present invention's protection.

1. A pharmaceutical intermediate, having a chemical structure shown in Formula II: 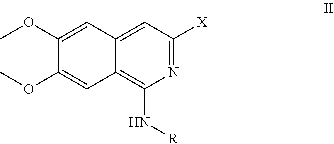
wherein X is a halogen, and R is an amino protecting group.

2. The pharmaceutical intermediate according to claim 1, wherein the amino protecting group is selected from benzyl, substituted benzyl, diphenylmethyl, substituted diphenylmethyl, triphenylmethyl, substituted triphenylmethyl, and tert-butoxycarbonyl.

3. A method for synthesizing a pharmaceutical intermediate according to claim 1, comprising reacting a compound shown in Formula I with an amino protecting agent R—NH₂ to obtain a compound shown in Formula II: 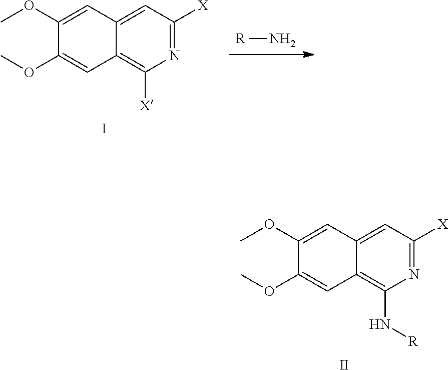
wherein X′ is a halogen.

4. The method according to claim 3, wherein R is selected from benzyl, substituted benzyl, diphenylmethyl, substituted diphenylmethyl, triphenylmethyl, substituted triphenylmethyl, and tert-butoxycarbonyl;
or, wherein the compound shown in Formula I is reacted with the amino protecting agent in a solvent selected from one or more of a group consisting of N-methylpyrrolidone, N,N-dimethylformamide, and dimethyl sulfoxide.

5. The method according to claim 3, wherein the compound shown in Formula I is reacted with the amino protecting agent in a solvent under a temperature of 100-160° C.;
or, wherein a mass/volume ratio of the compound shown in Formula I to the solvent is 1:(5-30) g/mL;
or, wherein a molar ratio of the compound shown in Formula I to the amino protecting agent is 1:(2-5);
or, wherein the method comprises a purification step, wherein a method for purification comprises adding water to the reaction product to quench reaction, followed by extracting quenched material, washing, drying, and evaporating the solvent from extracted organic phase to obtain a purified pharmaceutical intermediate.

6. A raw material compound, having a chemical structure shown in Formula a: 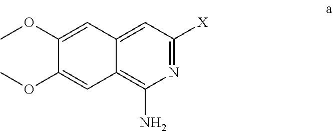
wherein X is a halogen.

7. A method of using the pharmaceutical intermediate of claim 1 for synthesizing isoquinoline derivatives, comprising reacting the compound shown in Formula I as a raw material to obtain a compound shown in Formula IV according to the following reaction route: 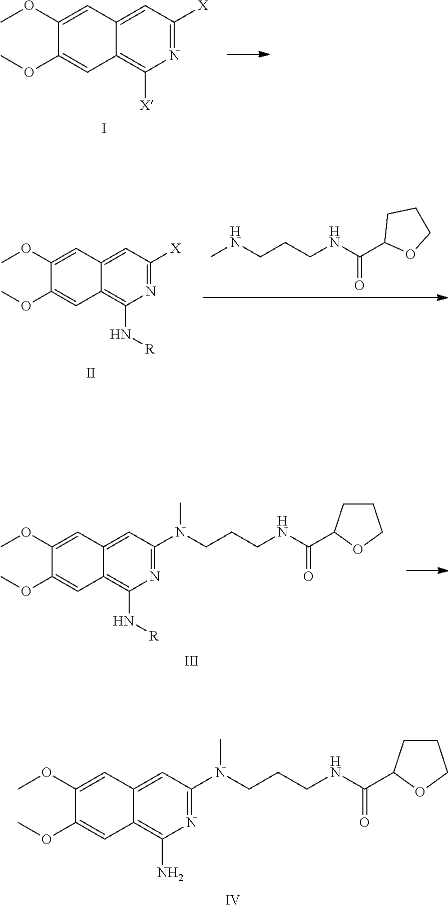
wherein, in preparing a compound shown in Formula III from the pharmaceutical intermediate shown in Formula II, a ligand, a catalyst, and a base are added for catalysis.

8. The method according to claim 7, wherein the catalyst used in preparing the compound shown in Formula III from the compound shown in Formula II is a palladium catalyst or a copper catalyst;
or, wherein the ligand used in preparing the compound shown in Formula III from the compound shown in Formula II is selected from one or more of a group consisting of PPh₃, P(o-tolyl)₃, P(t-Bu)₃, P(t-Bu)₃·HBF₄, PCy₃, n-BuP(Ad)₂, BINAP, Xantphos, DPEPhos, Dppf, CyPFt-Bu, Dppp, JohnPhos, CyJohnPhos, DavePhos, RuPhos, SPhos, Xphos, BrettPhos, t-BuXphos, t-BuBrettPhos, Me₄t-BuXphos, Bippyphos, MorDalPhos, and IPr HCl;
or, the base used in preparing the compound shown in Formula III from the compound shown in Formula II is sodium tert-butoxide, cesium carbonate, potassium tert-butoxide, potassium carbonate, potassium phosphate, lithium bis(trimethylsilyl)amide, 1,8-diazabicyclo[5.4.0]undec-7-ene, or 7-Methyl-1,5,7-triazabicyclo[4.4.0]dec-5-ene;
or, wherein a reaction temperature in preparing the compound shown in Formula III from the compound shown in Formula II is 90-130° C.;
or, wherein a solvent used in preparing the compound shown in Formula III from the compound shown in Formula II is an organic solvent with a boiling point higher than 90° C.

9. The method according to claim 7, wherein the compound shown in Formula II is reacted with tetrahydrofuran-2-carboxylicacid(3-methylamino-propyl)-amide in a solvent, a mass/volume ratio of the compound shown in Formula II to the solvent is 1:(5-100) g/mL;
or, wherein a molar ratio of the compound shown in Formula II to tetrahydrofuran-2-carboxylicacid(3-methylamino-propyl)-amide in preparing the compound shown in Formula III from the compound shown in Formula II is 1:(1-5);
or, wherein a molar ratio of the compound shown in Formula II to the catalyst in preparing the compound shown in Formula III from the compound shown in Formula II is 1:(0.01-0.5);
or, wherein a molar ratio of the compound shown in Formula II to the ligand in preparing the compound shown in Formula III from the compound shown in Formula II is 1:(0.02-1.0);
or, wherein a molar ratio of the compound shown in Formula II to the base in preparing the compound shown in Formula III from the compound shown in Formula II is 1:(1-4);
or, wherein a reaction temperature in preparing the compound shown in Formula IV from the compound shown in Formula III is 0-70° C.;
or, wherein a solvent used in preparing the compound shown in Formula IV from the compound shown in Formula III is trifluoroacetic acid, triethylsilane, methanesulfonic acid, trifluoromethanesulfonic acid, a mixture of trifluoroacetic acid and dichloromethane, a mixture of methanesulfonic acid and dichloromethane, or a mixture of triethylsilane and trifluoroacetic acid;
or, wherein a mass/volume ratio of the compound shown in Formula III to the solvent in preparing the compound shown in Formula IV from the compound shown in Formula III is 1:(5-100) g/mL.

10. An isoquinoline derivative, having a chemical structure shown in Formula IV:

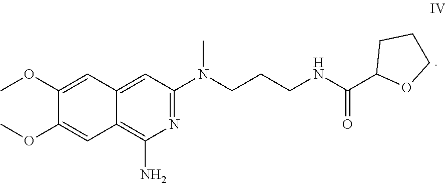 [SMILES: COc1cc2cc(N(C)CCCNC(=O)C3CCCO3)nc(N)c2cc1OC]

11. The pharmaceutical intermediate according to claim 1, wherein the amino protecting group is benzyl or substituted benzyl.

12. The pharmaceutical intermediate according to claim 1, wherein the amino protecting group is alkoxy-substituted benzyl or alkyl-substituted benzyl.

13. The method according to claim 3, wherein R is benzyl or substituted benzyl.

14. The method according to claim 3, wherein R is alkoxy-substituted benzyl or alkyl-substituted benzyl.

15. The method according to claim 7, wherein the catalyst used in preparing the compound shown in Formula III from the compound shown in Formula II is a palladium catalyst, and the palladium catalyst is selected from one or more of a group consisting of PdCl₂, Pd(PPh₃)₄, Pd(OAc)₂, Pd₂(dba)₃, Pd(dba)₂, PdCl₂(cod), [Pd(allyl)Cl]₂, PdCl₂ (CH₃CN)₂, Pd(acac)₂, Pd(PPh₃)₂Cl₂, Pd(Dppf)₂Cl₂, and PdCl₂[P(o-Tol)₃].

16. The method according to claim 7, wherein a solvent used in preparing the compound shown in Formula III from the compound shown in Formula II is toluene, 1,4-dioxane, N,N-dimethylformamide, or dimethyl sulfoxide.
